(19) United States
(12) Patent Application Publication
(10) Pub. No.: US 2013/0029996 A1
(43) Pub. Date: Jan. 31, 2013

(54) SPECIAL COMPOSITION FOR THE USE THEREOF AS A DRUG

(71) Applicants: Remy Burcelin, Toulouse Cedex 4 (FR); Eric Seree, Marseille Cedex 5 (FR)

(72) Inventors: Remy Burcelin, Toulouse Cedex 4 (FR); Eric Seree, Marseille Cedex 5 (FR)

(21) Appl. No.: 13/574,012

(22) PCT Filed: Jul. 28, 2011
(86) PCT No.: PCT/EP2011/050715

§ 371 Date: Oct. 2, 2012

(30) Foreign Application Priority Data
Jan. 21, 2010 (EP) 10305071.2

(51) Int. Cl. A61K 31/05; A61P 3/10; A61K 31/155; A61K 31/175; A61K 31/4985; A61P 9/04; A61P 19/02; A61P 25/28; A61P 25/16; A61P 25/00; A61P 9/12; A61P 9/10; A61P 3/00; A61P 29/00

(52) U.S. Cl. 514/249; 514/734; 514/635; 514/592

(57) ABSTRACT

The present invention relates to a composition containing at least one polyphenol, at least one polyethylene glycol and/or a functional equivalent and at least one glycol ether and/or a functional equivalent for application as a medicinal product.

The composition according to the invention can also be used as a food supplement or nutraceutical.

### TECHNICAL FIELD OF THE INVENTION

The present invention relates to a composition containing at least one polyphenol, at least one polyethylene glycol and/or a functional equivalent and at least one glycol ether and/or a functional equivalent for application as a medicinal product.

### PRIOR ART

The polyphenols, which are hydrophobic molecules, are compounds that occur naturally in plants of the spermatophyte class and particularly in grapevine. Such compounds, for example resveratrol, are found in grapes and in wine.

Among the polyphenols, there are the hydroxystilbenes. In the prior art the hydroxystilbenes are used among other things as depigmenting agents (JP87-192040), as vasodilators (EP 96-830517), as antithrombotic agents (JP 05016413), in the treatment of various cardiovascular disorders (CA 2187990), as inhibitors of mutagenesis and of carcinogenesis (JP 06024967), or are also described as antioxidants.

Resveratrol (3,4′,5-trihydroxystilbene) is a polyphenolic phytoalexin. This compound is synthesized by plants and acts as an antifungal in response to infections (Botrytis cinerea). Resveratrol occurs in various plants such as conifers, peanuts, the skin of red grapes, certain leguminous plants and Polygonum cuspidatum.

Resveratrol has therapeutic benefits that have long been known in Chinese and Japanese traditional medicine. Resveratrol has been implicated as being responsible for the decrease in cardiovascular risks called the “French Paradox”. In fact, a correlation has been established between consumption of red wine containing high levels of resveratrol, and decrease in coronary diseases. It has been demonstrated in numerous scientific studies that resveratrol is an antagonist of the receptor of the aryl hydrocarbons and of dioxin (AhR) (Casper, R. F. et al., Mol. Pharmacol. 1999, 56, 784-790).

Resveratrol also has antioxidant, anti-inflammatory, and osteoprotective activities and might have a preventive effect in certain cancers.

In-vivo models have been used for studying the absorption of resveratrol. In the rat, kinetic studies were conducted at different times after absorption of red wine. The results indicate that an absorption peak is detected 60 minutes after ingestion. After a short period, resveratrol is detected in the liver and kidneys (micromolar peak one hour after absorption). Elimination is very rapid by the kidneys as the preferential organ of elimination. Other studies show that serum resveratrol appears after 15 minutes and decreases very rapidly after 30 minutes. Similar studies were conducted in the mouse. Maximum absorption of resveratrol appears to occur in the duodenum and elimination of resveratrol is very rapid, with an absorption peak at 30 minutes.

It emerges from these studies that resveratrol is absorbed very rapidly, metabolized during the enterohepatic cycle and eliminated.

Numerous studies ex vivo and in vitro have focused on the absorption and metabolism of resveratrol to identify the localization of resveratrol in the various organs following its administration and the metabolites produced.

The ex vivo models are represented by perfusions of rat small intestine. This type of study shows that resveratrol is extracted from the small intestine at a level of 46%, with 21% being localized in the vessels and only 2% in the intestine. This resveratrol is 40% free, whereas 11% is glucurono-conjugated and 3% is in sulphated form. The glucuronoside form is the form that is present in the circulatory system, whereas the sulphated form is the form secreted in the intestinal lumen. This same type of study has been carried out in models of perfused ileum and colon.

The results indicate that only a tiny proportion of resveratrol is not metabolized. Studies on similar models of human liver or intestine indicate that resveratrol is also metabolized very rapidly. Resveratrol is therefore absorbed and metabolized in the intestine. When resveratrol passes into the circulation, it is largely carried by the serum proteins.

Studies in vitro using cell lines (intestinal line CaCo-2) have confirmed the results obtained on the perfused intestinal models.

Finally, since 2003, studies of metabolism and of bioavailability have been conducted in humans. The principal data indicate that resveratrol reaches a peak serum concentration after 30 minutes. Beyond 30 minutes, it is rare to find resveratrol that is unchanged and non-metabolized. In the best cases, 2% of resveratrol is found unchanged in the plasma.

All these data suggest that the activity of resveratrol as a therapeutic agent will be closely linked to its bioavailability, which results from its intestinal absorption and from the rapidity of its metabolism.

Thus, the poor results obtained with resveratrol as a therapeutic product are essentially due to its poor bioavailability.

The inventors have therefore used a pharmaceutical form that can increase the absorption of resveratrol via the epithelial cells and therefore its bioavailability.

Owing to this special pharmaceutical form of resveratrol, it has been possible to obtain surprising and spectacular results in various disorders such as metabolic or cardiovascular diseases.

### SUMMARY OF THE INVENTION

Thus, the invention relates to a composition for application as a medicinal product comprising at least one polyphenol, at least one polyethylene glycol and/or a functional equivalent and at least one glycol ether and/or a functional equivalent.

The composition according to the invention is preferably used for treating metabolic diseases, inflammatory diseases, neurodegenerative diseases, cardiovascular diseases and as prebiotic.

### DETAILED DESCRIPTION OF THE INVENTION

### Composition and Therapeutic Uses

The invention relates firstly to a composition for application as a medicinal product comprising:

a. at least one polyphenol;

b. at least one polyethylene glycol and/or a functional equivalent and;

c. at least one glycol ether and/or a functional equivalent.

In a preferred embodiment, the composition according to the invention is useful for treating metabolic diseases.

The term “metabolic diseases” means any type of nutritional disease or syndrome that disturbs the normal metabolism. Preferably, the metabolic disease according to the invention is a disorder of energy metabolism.

The term “disorder of energy metabolism” means a nutritional disease or syndrome in which the metabolism of energy-supplying nutrients (carbohydrates, lipids, proteins), is disturbed. These associated nutritional diseases and syndromes are for example diabetes, any hyperglycaemic syndrome, obesity, excess weight, undernourishment, malnutrition, cachexia.

In an even more preferred embodiment, the composition according to the invention can contain at least one other active principle.

This active principle can be an oral antidiabetic such as metformin, the sulphonylureas, glycosidase inhibitors, DPP4 inhibitors such as gliptins, thiazolidinediones or sulphamides.

In a preferred embodiment, the DPP4 inhibitor is sitagliptin, vildagliptin, saxagliptin, alogliptin, or some other molecule involved in the direct or indirect inhibition of the degradation of GLP-1.

In another preferred embodiment, the composition according to the invention is useful for treating inflammatory diseases.

The term “inflammatory diseases” means, for example, rheumatoid arthritis, spondylopathies, Crohn's disease, arthroses, arthropathies such as gonarthroses. The composition according to the invention can also be useful for post-operative inflammations, for example following the fitting of a prosthesis.

In another preferred embodiment, the composition according to the invention is useful for treating neurodegenerative diseases.

The term “neurodegenerative diseases” means for example Alzheimer's disease, Parkinson's disease, Huntington's disease, leukoaraiosis, progressive supranuclear paralysis, multiple scleroses or amyotrophic lateral sclerosis.

In another preferred embodiment, the composition according to the invention is useful for treating memory disorders and cognitive disorders. The composition can be used in the elderly for preventing the risk of loss of memory and of cognitive functions.

In another preferred embodiment, the composition according to the invention is useful for treating cardiovascular diseases.

The term “cardiovascular diseases” means for example hypertension, atherosclerosis, myocardial infarction, heart failure or aneurysm.

In another preferred embodiment, the composition according to the invention is useful for management of cardiometabolic risk factors.

The term “cardiometabolic risk” means change in the circulating concentration, relative to the standard values of healthy subjects, of lipids in all their forms: LDL, HDL, triglycerides, free fatty acids, in the form of lipid particles, glucose, inflammatory molecules, pro-oxidative molecules, platelet aggregation, nitric oxide and derivatives.

In another preferred embodiment, the composition according to the invention is useful as a prebiotic.

The term “prebiotic” means a molecule that modifies the intestinal flora so as to exert beneficial effects on health. Prebiotics notably affect metabolic and cardiovascular diseases, the risk of colon cancer, avitaminoses, osseous decalcification. The changes of the intestinal flora are notably characterized in response to prebiotics by increase in population of the genera bifidobacteria and lactobacteria.

In a preferred embodiment, the composition according to the invention can be used in the prevention of diseases such as metabolic and cardiovascular diseases.

In a preferred embodiment, the composition according to the invention is useful for treating neuromuscular diseases.

The term “neuromuscular diseases” means for example myopathies and amyotrophies.

In another preferred embodiment, the composition according to the invention can be used as a food supplement or nutraceutical.

The term “food supplement” means a foodstuff having a nutritional or physiological effect, marketed in the form of hard and soft capsules, pastilles, tablets, ampoules, infusions or oral solutions, the purpose of which is to supplement the usual human diet.

The term “nutraceutical” means a product that has a physiologically active dietary component that provides a health benefit (wellbeing as well as reduction of the risk of disease), independently of the basic nutritional characteristics.

In a preferred embodiment, the composition according to the invention contains at least one polyphenol, which can be a natural polyphenol or a synthetic polyphenol.

The term “synthetic polyphenol” means more specifically any polyphenol obtained by chemical synthesis and not by extraction from biological material (plants) as well as any derivative of a natural polyphenol modified by substitution or addition of atoms to the natural structure. Advantageously, these substitutions are halogens (Cl—, CF3-) or radicals of general structure R—O—, where R is an aliphatic chain or an aromatic ring or a nitrated radical.

In a preferred embodiment, the composition according to the invention contains at least one polyphenol that is a hydroxystilbene of formula (I),

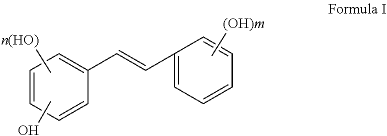

in which n is an integer between 0 and 4 inclusive and m is an integer between 0 and 5 inclusive. These compounds can be in a cis or trans form. According to the invention, the term hydroxystilbene covers both the compounds of formula I and their hydroxyalkylated derivatives.

Among the hydroxystilbenes, we may mention the mono-, di-, tri-, tetra-, penta-, hexa-, hepta-, octa-, and nonahydroxystilbenes, or their hydroxyalkylated derivatives, including for example 4′-hydroxystilbene, 2′,4′-dihydroxystilbene, 3′,4′-dihydroxystilbene, 4,4′-dihydroxystilbene, 2′,4′,4-trihydroxystilbene, 3′,4′,4-trihydroxystilbene, 2,4,4′-trihydroxystilbene, 3,4,4′-trihydroxystilbene, 3,4′,5-trihydroxystilbene, 2′,3,4-trihydroxystilbene, 2,3′,4-trihydroxystilbene, 2′,2,4′-trihydroxystilbene, 2,4,4′,5-tetrahydroxystilbene, 2′,3,4′,5-tetrahydroxystilbene, 2,2′,4,4′-tetrahydroxystilbene, 3,3′,4′,5-tetrahydroxystilbene, 2,3′,4,4′-tetrahydroxystilbene, 3,3′,4,4′-tetrahydroxystilbene, 3,3′,4′,5,5′-pentahydroxystilbene, 2,2′,4,4′,6-pentahydroxystilbene, 2,3′,4,4′,6-pentahydroxystilbene, 2,2′,4,4′,6,6′-hexahydroxystilbene.

Preferably, the composition according to the invention contains 3,4′,5-trihydroxystilbene or resveratrol.

In another preferred embodiment, the composition according to the invention contains at least one polyethylene glycol and/or a functional equivalent.

The term “functional equivalent” means, according to the invention, a compound which, when mixed with a polyphenol compound, exerts on the latter the same effects as a polyethylene glycol and/or a glycol ether.

Thus, according to the invention, it is possible to use a formulation comprising a polysorbate as functional equivalent of polyethylene glycol.

The term “polyethylene glycol” means any polymer corresponding to the formula H(OCH₂CH₂)ₙOH where n is greater than three. In this respect we may mention, as an example, the polyethylene glycols of average molecular weight between about 100 and 20000, preferably of average molecular weight between about 400 and about 10000, very preferably of average molecular weight between about 400 and about 600.

According to the invention, a polyethylene glycol of a given molecular weight can be used alone or mixed in all proportions with one or more other polyethylene glycols of different molecular weights or other functional equivalents.

The polyethylene glycols used in the context of the invention can be either in liquid form, or in semisolid form at room temperature, depending on their molecular weight. Consequently, these polymers will be selected appropriately according to whether the formulation that is intended to improve the absorption of polyphenols according to the invention is to be in liquid form or conversely in semisolid form.

The composition according to the invention can comprise polyethylene glycol, and/or a functional equivalent thereof, for example a polysorbate, in a proportion between 20 and 97 wt. %, preferably between 40 and 97 wt. % of the total weight of the composition.

In another preferred embodiment, the composition according to the invention contains at least one glycol ether and/or a functional equivalent.

According to the invention, glycerol or polyglyceryl-3-dioleate (polyglyceryl ester of fatty acids or an equivalent thereof) can be used as a functional equivalent of glycol ether.

According to the invention, the glycol ether can be selected from diethyleneglycol ethers such as for example diethyleneglycol alkyl ethers, in particular the (C1-C4) alkyl ethers of diethyleneglycol, selected from the methyl ether of diethyleneglycol, the ethyl ether of diethyleneglycol, the propylyl ethers of diethyleneglycol or the butyl ethers of diethyleneglycol, very particularly the mono-(C1-C4) alkyl ethers of diethyleneglycol selected from monomethyl ether of diethyleneglycol, monoethyl ether of diethyleneglycol, monopropylyl ethers of diethyleneglycol or monobutyl ethers of diethyleneglycol.

Among the diethyleneglycol alkyl ethers, the methyl and ethyl ethers, notably diethyleneglycol monoethyl ether, are preferred.

According to the invention, the glycol ethers can be used alone or combined in all proportions with one or more other glycol ether(s) or other functional equivalents.

The composition according to the invention can comprise glycol ether, and/or a functional equivalent thereof, for example glycerol or polyglyceryl-3-dioleate (polyglycerol ester of fatty acids or an equivalent thereof), in a proportion between 2 and 79 wt. %, preferably between 2 and 59 wt. % of the total weight of the composition.

A particularly preferred composition according to the invention will comprise between 50 and 93% of polyethylene glycol, and/or a functional equivalent thereof, for example a polysorbate, between 3 and 46% of glycol ether, and/or a functional equivalent thereof, for example glycerol or polyglyceryl-3-dioleate (polyglycerol ester of fatty acids or an equivalent thereof), and a sufficient amount of water to reach 100%.

In another preferred embodiment, the composition according to the invention can further comprise at least one emulsifier. Advantageously according to the invention the emulsifier can be a polysorbate, even more advantageously a polysorbate selected from Polysorbate 20 (Tween 20 or polyoxyethylene (20) sorbitan monolaurate), Polysorbate 40 (Tween 40 or polyoxyethylene (20) sorbitan monopalmitate), Polysorbate 60 (Tween 60 or polyoxyethylene (20) sorbitan monostearate), or Polysorbate 80 (Tween 80 or polyoxyethylene (20) sorbitan monooleate).

In another preferred embodiment, the composition according to the invention is suitable for administration by the oral route, the nasal route or the rectal route.

The composition according to the invention can thus be in the ileum of sugar-coated pills, hard or soft capsules, gels, emulsions, tablets, or some other pharmaceutical form for oral administration. The composition according to the invention can also be in the form of a nasal spray or a suppository. These forms are produced by the usual methods known by a person skilled in the art.

According to a particular embodiment of the invention, the composition according to the invention can be formulated in an encapsulated form so as to improve the lifetime significantly.

Other features and advantages of the invention will be understood more clearly from the following examples, given for purposes of illustration and non-limiting.

### FIGURES

FIG. 1: Effect of the Resveratrol-Containing Composition According to the Invention on Glucose Tolerance.

NC corresponds to the controls. These mice were fed the diabetogenic diet without the resveratrol-containing composition according to the invention. RSV corresponds to the mice that were fed the diabetogenic diet supplemented with the resveratrol-containing composition according to the invention.

FIG. 2: Effect of the Resveratrol-Containing Composition According to the Invention on GLP-1 Synthesis.

HFD corresponds to the controls. These mice were fed the diabetogenic diet without the resveratrol-containing composition according to the invention. HFD RSV corresponds to the mice that were fed the diabetogenic diet supplemented with the resveratrol-containing composition according to the invention.

FIG. 2 A corresponds to analyses of the mRNA of active GLP-1 in the colon, FIG. 2 B to the analyses of the active GLP-1 protein in the colon, and FIG. 2 C to the analyses of the molar concentration of active GLP-1 in the hepatic portal vein.

FIG. 3: Effect of the Resveratrol-Containing Composition According to the Invention on Glucose Tolerance in Knockout Mice for the Active GLP-1 Receptor.

HFD corresponds to the controls. These mice were fed the diabetogenic diet without the resveratrol-containing composition according to the invention. HFD RSV corresponds to the mice that were fed the diabetogenic diet supplemented with the resveratrol-containing composition according to the invention.

FIG. 4: Effect of the Resveratrol-Containing Composition According to the Invention on the Circulating Insulin Concentration.

NC corresponds to the controls. These mice were fed the diabetogenic diet without the resveratrol-containing composition according to the invention. RSV corresponds to the mice that were fed the diabetogenic diet supplemented with the resveratrol-containing composition according to the invention.

FIG. 5: Effect of the Resveratrol-Containing Composition According to the Invention on Secretion of IL10.

NC corresponds to the controls. These mice were fed the inflammatory diabetogenic diet without the resveratrol-containing composition according to the invention. HFD RSV or RSV corresponds to the mice that were fed the inflammatory diabetogenic diet supplemented with the resveratrol-containing composition according to the invention.

FIG. 5 A corresponds to analyses of the mRNA of IL10 in the colon, FIG. 5 B to analyses of the protein IL10 in the colon, and FIG. 5 C to analyses of the mRNA of IL10 in the liver.

FIG. 6: Effect of the Resveratrol-Containing Composition According to the Invention on PAI-1 in the Hypothalamus.

NC corresponds to the controls. These mice were fed the inflammatory diabetogenic diet without the resveratrol-containing composition according to the invention. RSV corresponds to the mice that were fed the inflammatory diabetogenic diet supplemented with the resveratrol-containing composition according to the invention.

FIG. 7: Effect of the Resveratrol-Containing Composition According to the Invention on IL10 in the Hypothalamus.

NC corresponds to the controls. These mice were fed the inflammatory diabetogenic diet without the resveratrol-containing composition according to the invention. RSV corresponds to the mice that were fed the inflammatory diabetogenic diet supplemented with the resveratrol-containing composition according to the invention.

FIG. 8: Testing in the Y-Shaped Maze.

YM corresponds to young mice (4 months) and EM corresponds to elderly mice (22 months). The sign + corresponds to the mice that received the resveratrol-containing composition according to the invention. The sign − corresponds to mice that did not receive the resveratrol-containing composition according to the invention.

FIG. 9: Effect of the Resveratrol-Containing Composition According to the Invention on the Lipid Risk Factor Parameters Associated with Cardiovascular and Metabolic Risk.

NC corresponds to the controls. These mice were fed the inflammatory diabetogenic diet without the resveratrol-containing composition according to the invention. RSV corresponds to the mice that were fed the inflammatory diabetogenic diet supplemented with the resveratrol-containing composition according to the invention.

FIG. 10: Prebiotic Effect of the Resveratrol-Containing Composition According to the Invention.

In FIG. 10 A, NC corresponds to the controls. These mice were fed the standard laboratory diet without the resveratrol-containing composition according to the invention. HFD corresponds to mice fed an inflammatory diabetogenic diet only.

In FIG. 10 B, HFD corresponds to the controls. These mice were fed the inflammatory diabetogenic diet without the resveratrol-containing composition according to the invention. HFD RSV corresponds to mice fed the inflammatory diabetogenic diet with the resveratrol-containing composition according to the invention.

FIG. 11: Co-Administration of Sitagliptin, a Dipeptidyl Peptidase-4 Inhibitor, and BioA-RSV (Resveratrol in the Optimized Pharmaceutical Form), which Improves Glucose Tolerance in a Model of Diabetes Induced in the Mouse with a High-Fat Diet.

A) Glycaemia profiles (mg/dL) of diabetic mice treated with BioA-RSV (squares) or with sitagliptin combined with BioA-RSV (triangles) for five weeks; B) Concentration of active GLP-1 in the portal blood (pM) and C) relative expression level (REL) of mRNA in diabetic mice treated with BioA-RSV (black bar) and sitagliptin combined with BioA-RSV (dotted bar) for five weeks. The data are presented as mean value+SEM in 8 mice per group. * and *** indicate significant differences between the groups when p<0.05 and p<0.001, respectively, after applying the Student t-test.

### EXAMPLES

### Example 1

### Application of the Composition According to the Invention in Metabolic Diseases

A—Effect on Glucose Tolerance:

Materials & Method

The mice are treated with an inflammatory diabetogenic diet, inducing type 2 diabetes, for 5 weeks. They are then treated with the composition according to the invention supplemented or not with resveratrol and the mice are injected by the oral route with glucose in order to test glucose tolerance, which is an indicator of the diabetic state.

Results

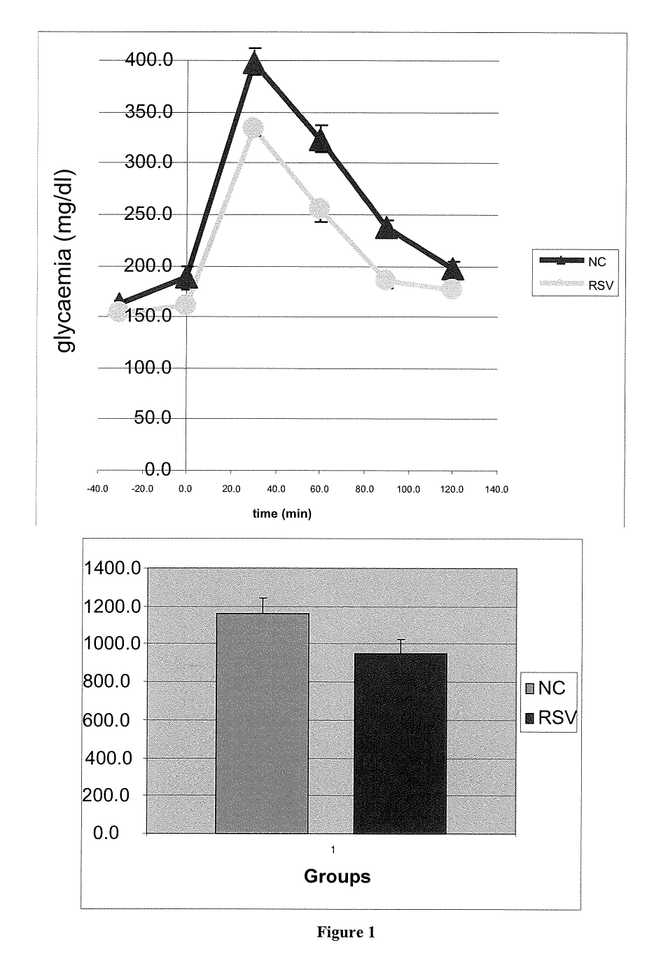

FIG. 1 shows that glycaemia is greatly reduced owing to the composition according to the invention supplemented with resveratrol.

B—Effect on Synthesis of GLP-1:

Materials & Method

The mice are treated with an inflammatory diabetogenic diet, inducing type 2 diabetes, for 5 weeks. They are then treated with the composition according to the invention supplemented or not with resveratrol.

Results

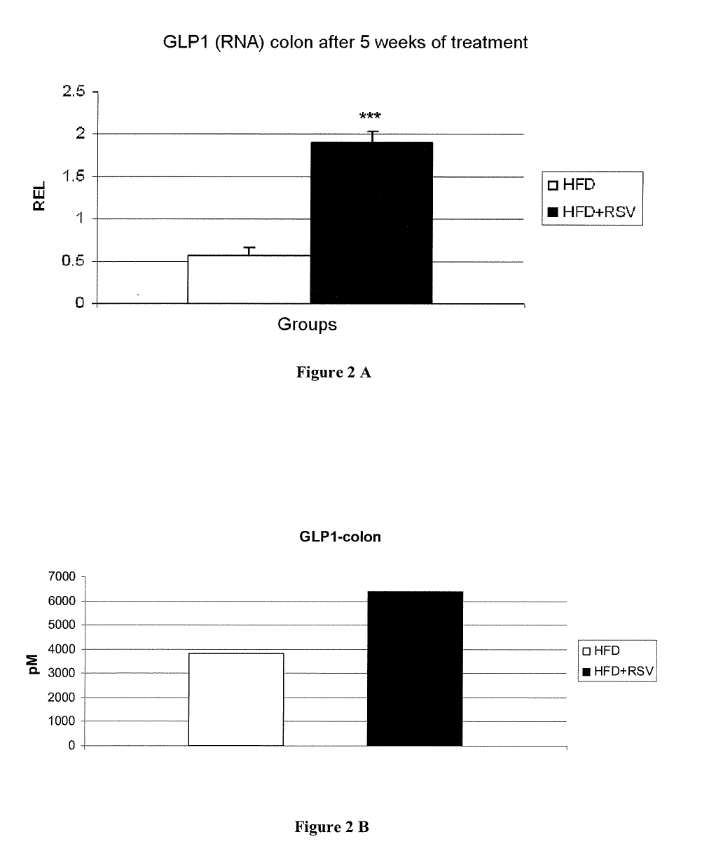

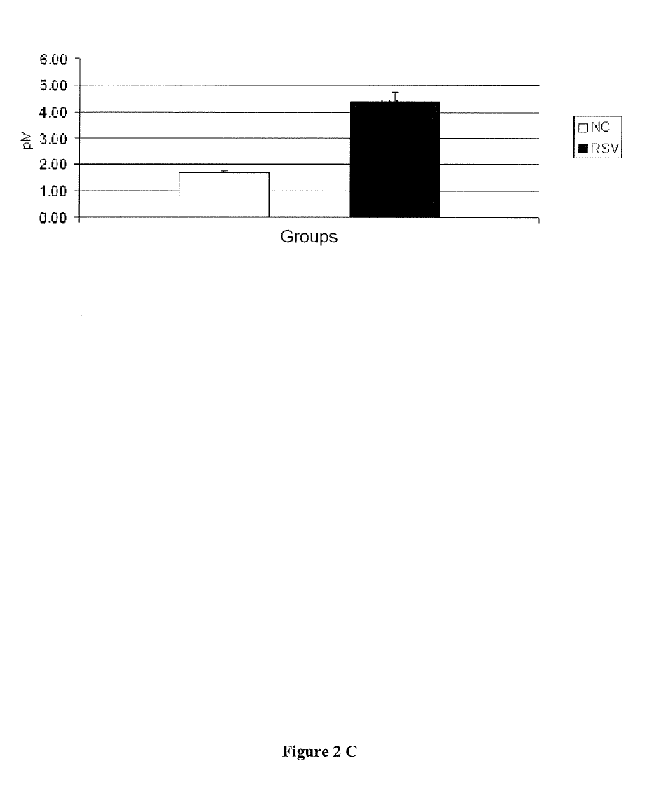

FIG. 2 shows the effect of the resveratrol-containing composition according to the invention on GLP-1 synthesis. GLP-1 is an intestinal hormone that is secreted during a meal and increases the secretion of insulin and decreases hyperglycaemia. The mRNA and the GLP1 protein are greatly increased in the colon (FIGS. 2.A. and 2.B.) and the molar concentration of active GLP1 is also increased in the hepatic portal vein (FIG. 2.C.).

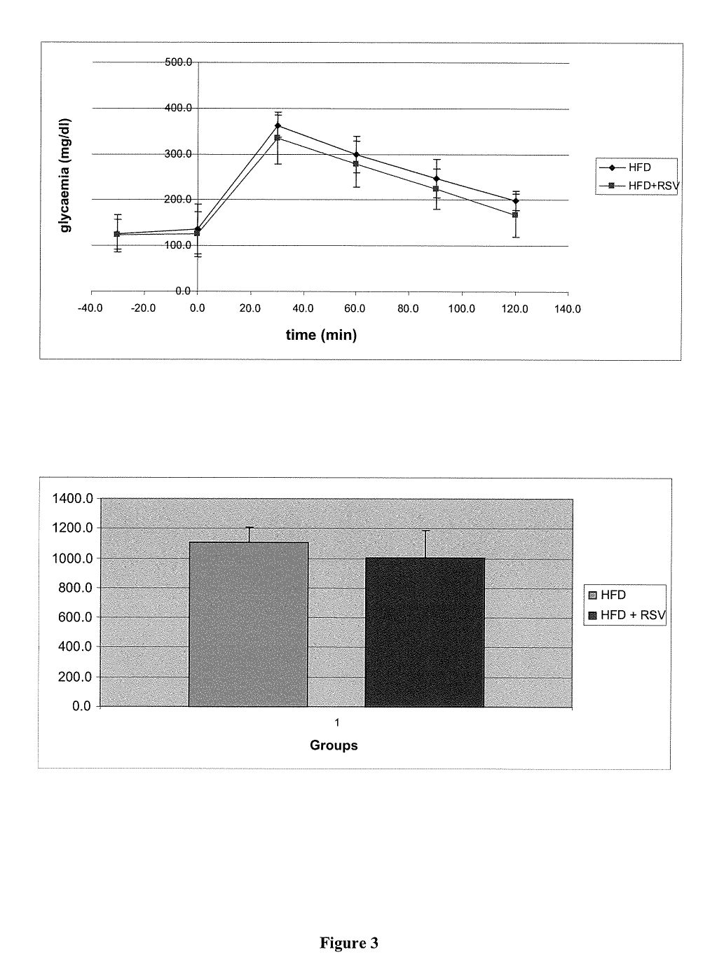

FIG. 3 shows that no therapeutic effect of the resveratrol-containing composition according to the invention is obtained in mice in which the receptor specific to active GLP-1 was removed genetically by genetic engineering. The anti-diabetogenic effect of RSV therefore requires the action of GLP-1 on its receptor.

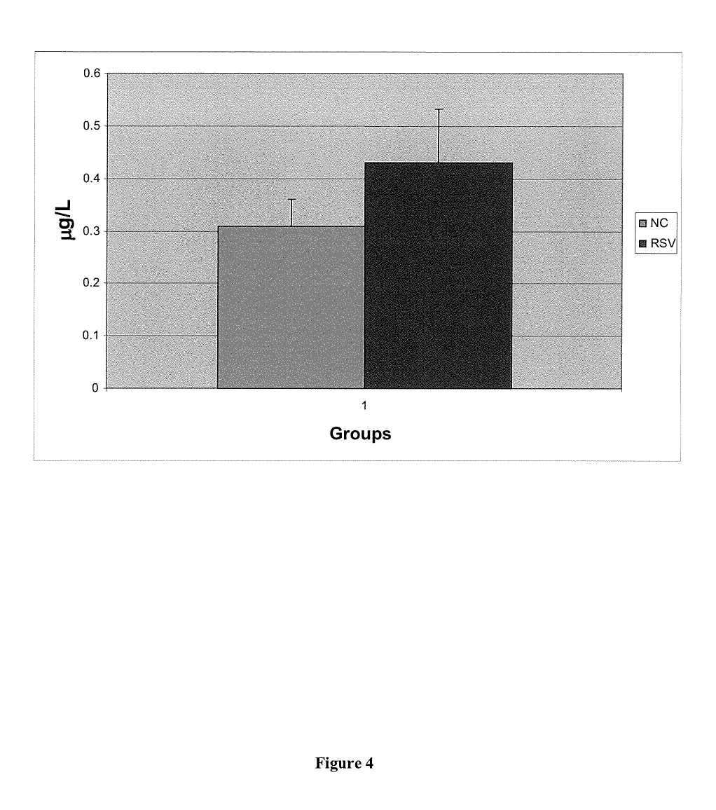

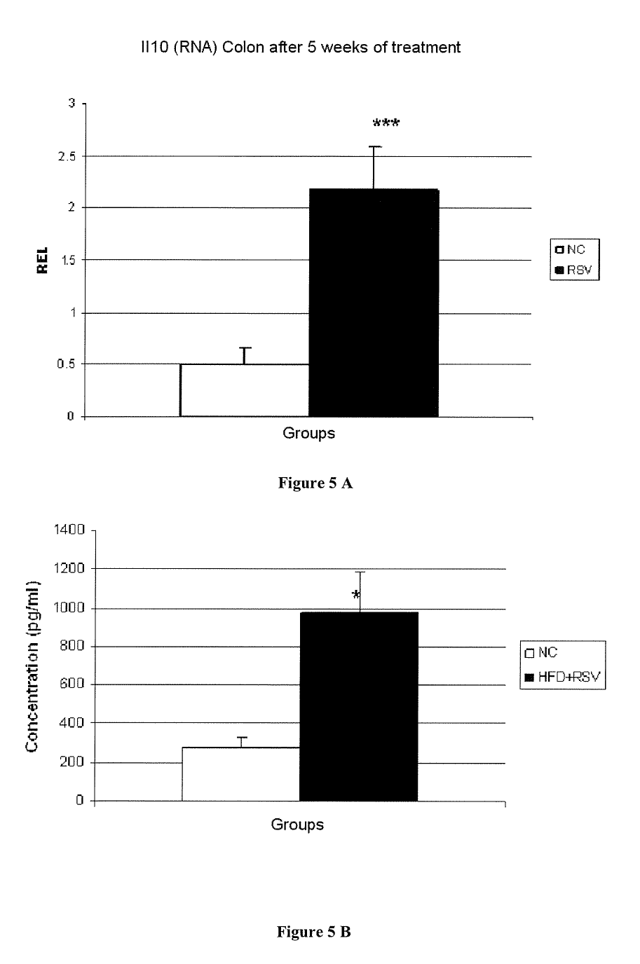

FIG. 4 shows that the resveratrol-containing composition according to the invention increases insulin secretion. This effect requires GLP-1.

### Example 2

### Application of the Composition According to the Invention in Inflammatory Diseases

Materials & Method

The mice are treated with a fatty diet supplemented or not with the resveratrol-containing composition according to the invention (0.04% W/W). After 5 weeks, the mice are sacrificed and the mRNAs and the protein corresponding to the principal anti-inflammatory cytokine IL10 are measured in the colon and the liver and the mRNA of the marker PAI-1 is measured in the hypothalamus.

Results

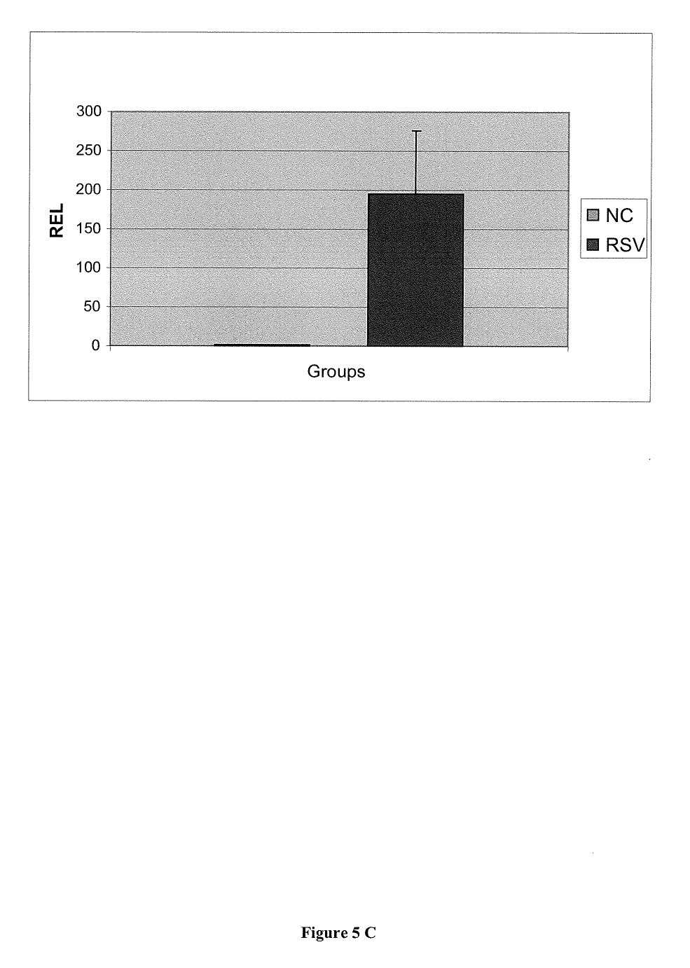

Interleukin 10 is an anti-inflammatory molecule secreted by certain blood cells (such as the monocytes). FIGS. 5.A. and 5.B. show an increase in mRNA and the protein IL10 in the colon. FIG. 5.C. shows an increase in the mRNA of IL10 in the liver. These results demonstrate the anti-inflammatory effect of the resveratrol-containing composition according to the invention.

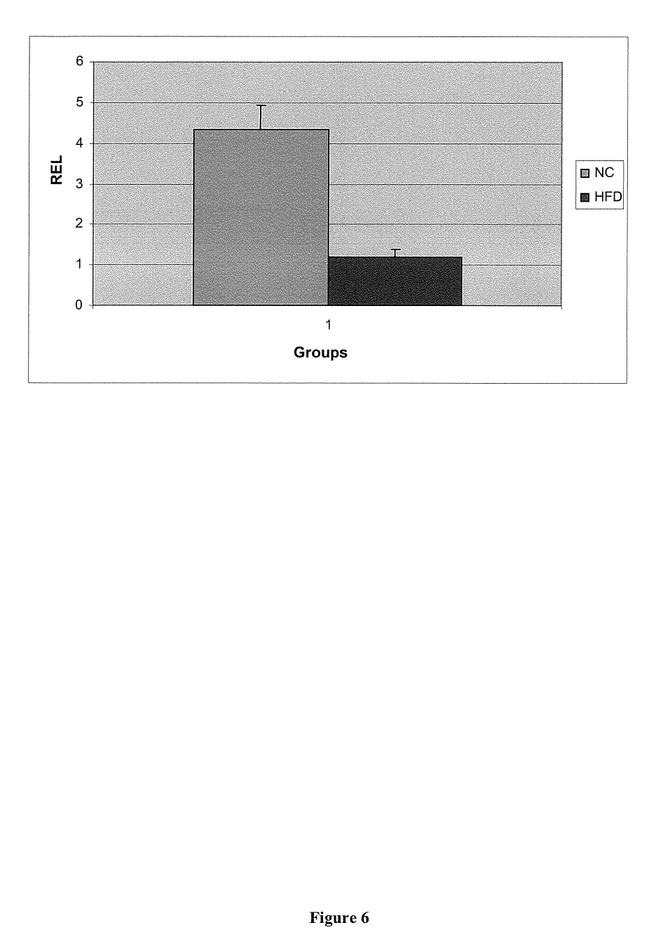

FIG. 6 shows the effect of the resveratrol-containing composition according to the invention on PAI1 in the hypothalamus. PAI-1 is the best marker for inflammation in metabolic diseases (such as diabetes, obesity). It is regarded as a pro-inflammatory marker. The resveratrol-containing composition according to the invention therefore makes it possible to reduce the mRNA of PAI-1 in the hypothalamus.

### Example 3

### Application of the Composition According to the Invention in Neurodegenerative Diseases

A—Effect on Synthesis of IL10:

Materials & Method

Mice aged 8 weeks are treated for 5 weeks with an inflammation-inducing and diabetogenic diet, which has the effect of inducing an inflammatory state and diabetes and promoting neurodegeneration. With the hypothalamus as a control of the metabolic inflammatory reaction, the mRNAs coding for the anti-inflammatory IL10 were measured.

Results

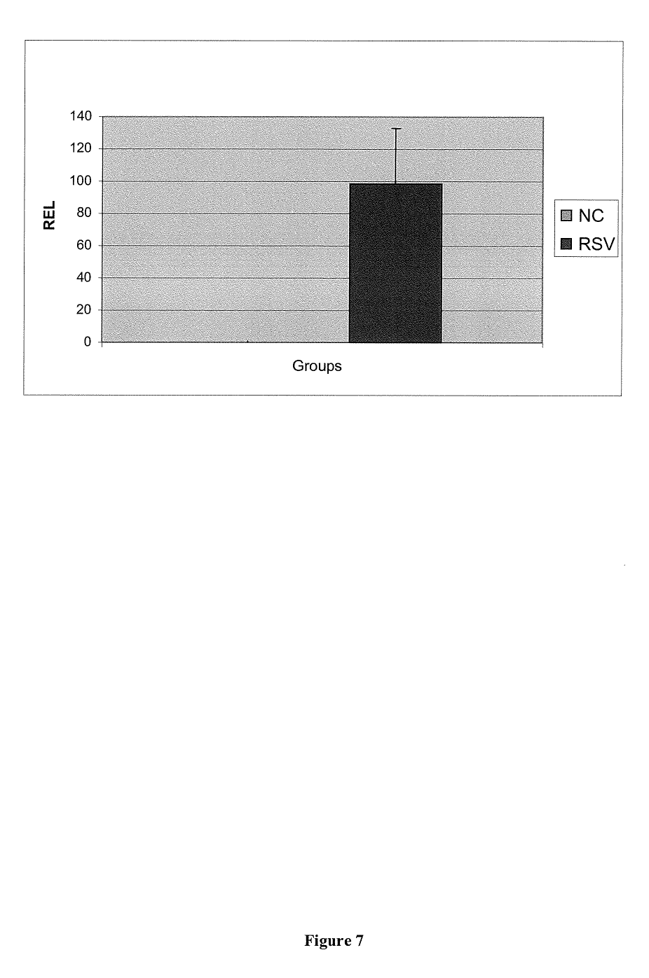

FIG. 7 shows an increase in the mRNA of IL10 in the hypothalamus. These results therefore demonstrate the anti-inflammatory effect of the resveratrol-containing composition according to the invention.

B—Testing in the Y-Shaped Maze:

Materials & Method

Elderly mice, liable to develop neurodegeneration, are used for this. These mice are fed a standard diet supplemented or not with the resveratrol-containing composition according to the invention.

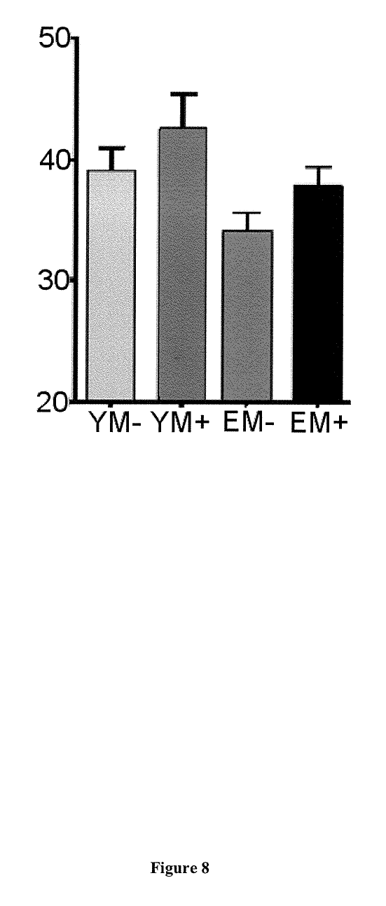

FIG. 8 shows the effect of the resveratrol-containing composition according to the invention on testing in the Y-shaped maze. Administration of this composition to the elderly mice produces a statistically significant increase in the entry percentage (p<0.05), which suggests an improvement in memory and cognition.

### Example 4

### Application of the Composition According to the Invention in Cardiovascular Diseases and Prevention of Cardiometabolic Risk Factors

Materials & Method

The mice are treated with a fatty diet inducing diabetes and inflammation for 5 weeks. The levels of triglycerides and LDL cholesterol and glycaemia are measured.

Results

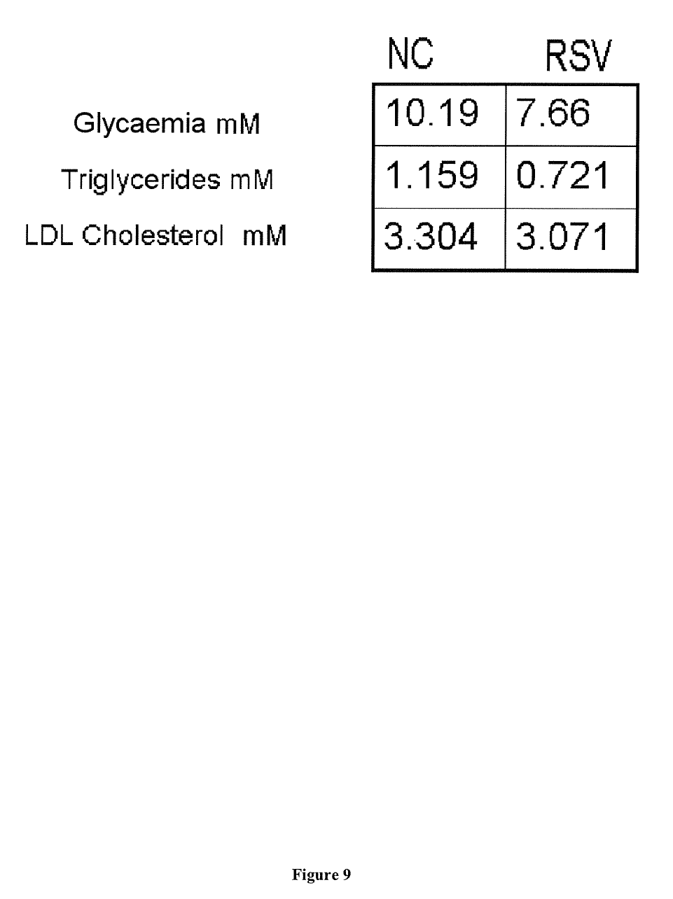

FIG. 9 shows the effect of the resveratrol-containing composition according to the invention. Various parameters associated with cardiovascular risk (glycaemia, triglycerides and LDL cholesterol) are reduced considerably. Thus, the risk of developing a cardiovascular disease is greatly reduced. There is also a decrease in glycaemia and therefore in the risk of becoming diabetic.

### Example 5

### Application of the Composition According to the Invention as Prebiotic

Materials & Method

The mice are treated with or without a fatty diet inducing diabetes and inflammation and with or without the resveratrol-containing composition according to the invention.

After 5 weeks the mice are sacrificed and the bacterial DNA16S of the colon are extracted. Using PCR and denaturing gradient gel, the diversity of the different bacterial species present in the intestine is demonstrated. A Pearson tree analysis makes it possible to form clusters corresponding to the various species and position the individuals relative to their proximity of metagenomic similarity (bacterial genome).

The individuals that are most similar from the standpoint of diversity of the intestinal flora are closest on the Pearson tree.

Results

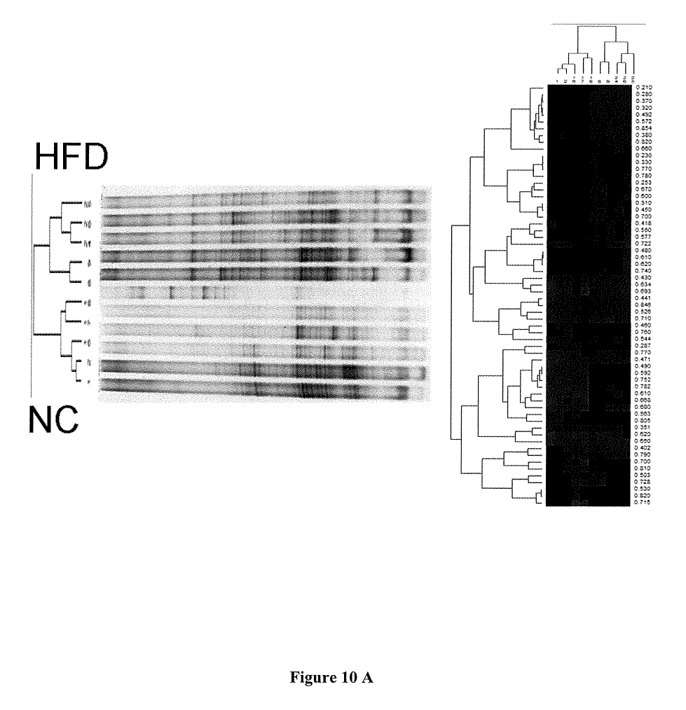

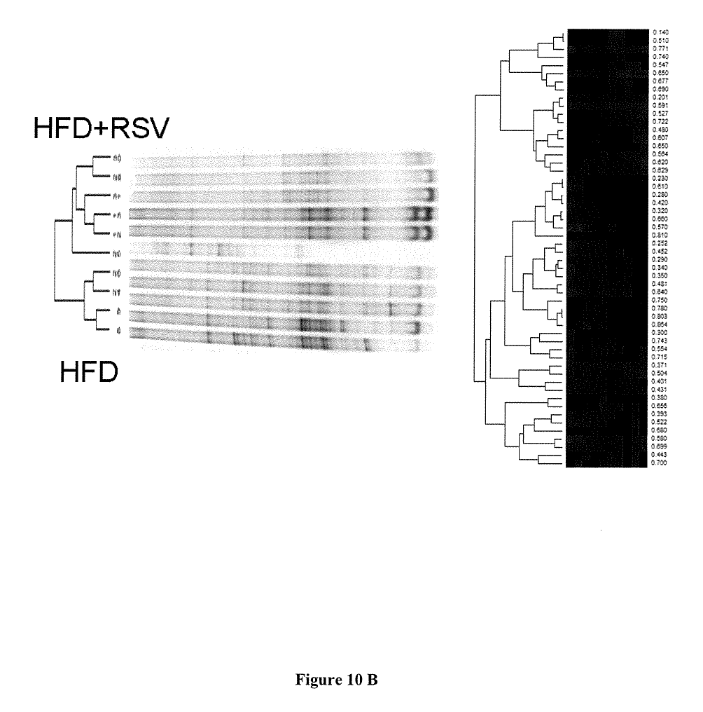

FIG. 10.A. shows that two groups of mice are formed. All of the control mice fed the standard laboratory diet were combined when forming clusters by Pearson tree analysis. Therefore the same applies to the mice fed the fatty diet. The fatty diet therefore induces a change in intestinal flora characteristic of each group and therefore identifiable by this PCR analysis.

FIG. 10.B. shows that two groups of mice are formed. All of the control mice fed the fatty diet not supplemented with the resveratrol-containing composition according to the invention were combined when forming clusters by Pearson tree analysis. Therefore the same applies to the mice fed the fatty diet supplemented with the resveratrol-containing composition according to the invention. Supplementation therefore certainly induces a change in intestinal flora. There is therefore clearly a prebiotic effect, since the intestinal flora is altered, and this is beneficial for health.

### Example 6

### Application of the Composition According to the Invention in Combination with a Dipeptidyl Peptidase-4 Inhibitor

Materials & Method

Eight-week-old male C57BL/6J mice (Charles River, L'Arbresle, France) were housed in strict sanitary conditions (germ-free) with a (12/12) cycle of light and darkness (light 10 PM/darkness 10 AM). The mice have free access to water and food. The mice are subjected to a normal diet or a high-fat diet for five weeks. This high-fat diet induces diabetes before obesity. One group of mice was treated with either resveratrol in active pharmaceutical form (BioA-RSV) combined or not with sitagliptin (5 mg daily in the feed). All the animal experiments were approved by the local ethics committee of the Rangueil CHU (University Hospital Centre) in Toulouse.

Results

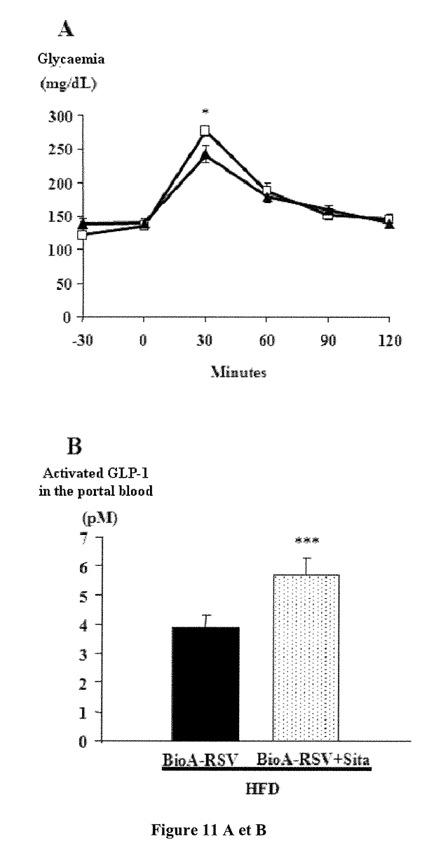

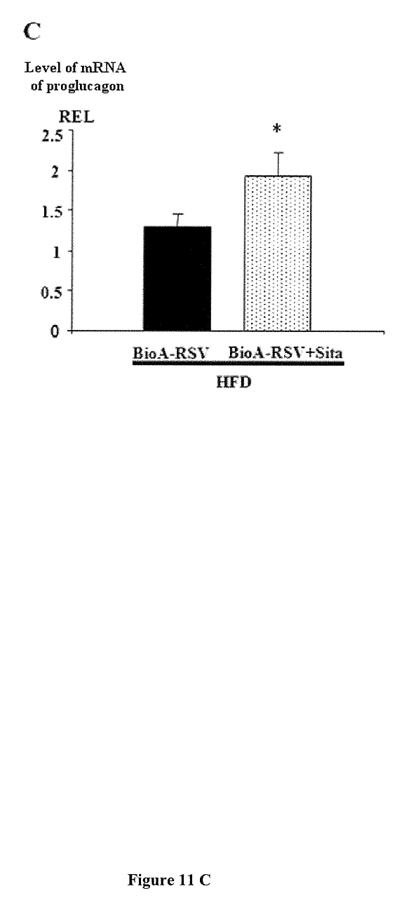

Our results show that administration of the combination of resveratrol and sitagliptin makes it possible to decrease glycaemia more effectively than administration of resveratrol alone 30 minutes after the oral dose of glucose. (FIG. 11A). The same applies regarding the portal concentration of GLP-1 (FIG. 11B) and the intraintestinal concentration of proglucagon precursor of GLP-1 (FIG. 11C).

We claim:

1-12. (canceled)

13. A pharmaceutical composition or probiotic comprising
a) at least one polyphenol;
b) at least one polyethylene glycol and/or a functional equivalent thereof; and
c) at least one glycolic ether and/or a functional equivalent thereof.

14. The pharmaceutical composition or probiotic of claim 13, wherein said at least one polyphenol is resveratrol.

15. The pharmaceutical composition or probiotic of claim 13, wherein said polyethylene glycol and/or a functional equivalent thereof is a polymer corresponding to the formula H(OCH₂CH₂)ₙOH wherein n is greater than 3.

16. The pharmaceutical composition or probiotic of claim 13, wherein said glycolic ether and/or a functional equivalent is a diethyleneglycol ether.

17. The pharmaceutical composition or probiotic of claim 16, wherein said diethyleneglycol ether is a diethyleneglycol alkyl ether.

18. The pharmaceutical composition or probiotic of claim 17, wherein said diethyleneglycol alkyl ether is a (C1-C4) diethyleneglycol alkyl ether.

19. The pharmaceutical composition or probiotic of claim 18, wherein said (C1-C4) diethyleneglycol alkyl ether is a mono(C1-C4) diethyleneglycol alkyl ether.

20. The pharmaceutical composition or probiotic of claim 19, wherein said mono(C1-C4) diethyleneglycol alkyl ether is diethyleneglycol monoethyl ether.

21. A method of treating a metabolic disease in a patient in need thereof, comprising
administering to said patient a therapeutically effective amount of a pharmaceutical composition comprising
a) at least one polyphenol;
b) at least one polyethylene glycol and/or a functional equivalent thereof; and
c) at least one glycolic ether and/or a functional equivalent thereof.

22. The method of claim 21, wherein said metabolic disease is diabetes.

23. The method of claim 22, wherein said pharmaceutical composition further comprises at least one other active principle selected from other group consisting of metformin, a sulphonylurea, a glycosidase inhibitor, and a DPP4 inhibitor.

24. The method of claim 23, wherein said DPP4 inhibitor is a gliptin, a thiazolidinedione or a sulphamide.

25. The method of claim 24, wherein said gliptin is selected from the group consisting of sitaglipin, vildagliptin, saxagliptin, alogliptin, and a molecule implicated in direct or indirect inhibition of degradation of GLP-1.

26. The method of claim 25, wherein said gliptin is sitaglipin.

27. A method of treating an inflammatory disease in a patient in need thereof, comprising
administering to said patient a therapeutically effective amount of a pharmaceutical composition comprising
a) at least one polyphenol;
b) at least one polyethylene glycol and/or a functional equivalent thereof; and
c) at least one glycolic ether and/or a functional equivalent thereof.

28. The method of claim 27, wherein said inflammatory disease is selected from the group consisting of rheumatoid arthritis, spondylopathy, Crohn's disease, arthrosis, and anthropathy.

29. The method of claim 28, wherein said anthropathy is gonarthroses.

30. A method of treating an neurodegenerative disease in a patient in need thereof, comprising
administering to said patient a therapeutically effective amount of a pharmaceutical composition comprising
a) at least one polyphenol;
b) at least one polyethylene glycol and/or a functional equivalent thereof; and
c) at least one glycolic ether and/or a functional equivalent thereof.

31. The method of claim 30, wherein said neurodegenerative disease is selected from the group consisting of Alzheimer's disease, Parkinson's disease, Huntington's disease, leukoaraiosis, progressive supranuclear paralysis, multiple sclerosis and amyotrophic lateral sclerosis.

32. A method of managing cardiometabolic risk factors and/or treating a cardiovascular disease in a patient in need thereof, comprising
administering to said patient a therapeutically effective amount of a pharmaceutical composition comprising
a) at least one polyphenol;
b) at least one polyethylene glycol and/or a functional equivalent thereof; and
c) at least one glycolic ether and/or a functional equivalent thereof.

33. The method of claim 32, wherein said cardiovascular disease is selected from the group consisting of hypertension, atherosclerosis, myocardial infarction, heart failure and aneurysm.

34. A method of modifying intestinal flora in order to exert a beneficial effect on the health of a subject in need thereof, comprising
administering to said subject a therapeutically effective amount of a prebiotic composition comprising
a) at least one polyphenol;
b) at least one polyethylene glycol and/or a functional equivalent thereof; and
c) at least one glycolic ether and/or a functional equivalent thereof.
